(19) United States
(12) Patent Application Publication
(10) Pub. No.: US 2025/0255482 A1
(43) Pub. Date: Aug. 14, 2025

(54) DIGITAL LIGHT PROCESSING (DLP) FOR FUNDUS IMAGING

(71) Applicant: Identifeye Health Inc., Guilford, CT (US)

(72) Inventor: Georg Schuele, Portola Valley, CA (US)

(21) Appl. No.: 19/052,212

(22) Filed: Feb. 12, 2025

Related U.S. Application Data
(60) Provisional application No. 63/552,726, filed on Feb. 13, 2024.

(51) Int. Cl. A61B 3/12; A61B 3/14

(52) U.S. Cl. CPC A61B 3/12; A61B 3/14

(57) ABSTRACT

Some aspects relate to techniques for imaging a fundus of a subject's eye using an apparatus comprising, an illumination source, a digital micromirror device (DMD), a fundus imaging device, and a processor.

### CROSS-REFERENCE TO RELATED APPLICATIONS

This application claims the benefit of priority under 35 U.S.C. § 119(e) of U.S. Provisional Patent Application 63/552,726, titled “ADAPTIVE STRUCTURED ILLUMINATION FOR OCULAR IMAGING,” filed Feb. 13, 2024, which is incorporated by reference herein in its entirety.

### BACKGROUND

Techniques for imaging and/or measuring a subject's eye would benefit from improvement.

### SUMMARY

Some aspects of the present disclosure relate to a system comprising: a plurality of imaging components comprising: an illumination source configured to emit light of a plurality of colors including a first color; a digital micromirror device (DMD) configured to project illumination patterns onto a subject's eye using the light emitted from the illumination source; at least one image sensor; and a processor configured to perform a method for multispectral imaging, the method comprising: transmitting instructions to one or more of the plurality of imaging components configured to cause the one or more of the plurality of imaging components to adjust an exposure to light of the first color relative to exposure to light of other colors included in the plurality of colors.

Some aspects of the present disclosure relate to a system comprising: an illumination source configured to emit light; a digital micromirror device (DMD) configured to project, during an imaging period, a plurality of illumination patterns onto a subject's eye using the light emitted from the illumination source, the imaging period starting at an initial time point corresponding to a projection of an initial illumination pattern onto the subject's eye and ending at a final time prior to a time of constriction of a pupil of the subject's eye; a fundus imaging device configured to capture a plurality of images of a fundus of the subject's eye, each image in the plurality of images captured during projection of a respective illumination pattern of the plurality of illumination patterns; and a processor configured to generate an image of the fundus using the plurality of images.

Some aspects of the present disclosure relate to a method for imaging a fundus of a subject's eye using a fundus imaging system, the fundus imaging system comprising a fundus image sensor comprising a plurality of pixels and employing a rolling shutter and a digital micromirror device (DMD), the method comprising: capturing an image of the fundus of the subject's eye at least in part by: projecting a first illumination pattern onto a first portion of a subject's eye using the DMD; and while projecting the first illumination pattern onto the first portion of the subject's eye, capturing image data depicting the first portion of the subject's eye using the rolling shutter and a first subset of the pixels of the fundus image sensor.

Some aspects of the present disclosure relate to a system comprising: at least one illumination source configured to emit light; a digital micromirror device (DMD) configured to project a fixation target onto a subject's fundus using at least some of the light emitted from the illumination source; an imaging device configured to capture an image of the subject's eye while the fixation target is projected onto the subject's fundus; and a processor configured to transmit instructions to the DMD configured to cause the DMD to vary the fixation target.

The foregoing summary is not intended to be limiting. Moreover, various aspects of the present disclosure may be implemented alone or in combination with other aspects.

### BRIEF DESCRIPTION OF DRAWINGS

Various aspects and embodiments of the disclosure provided herein are described below with reference to the following figures. The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A is a schematic view of an example fundus imaging system, according to a first embodiment.

FIG. 1B is a schematic view of an example fundus imaging system, according to a second embodiment.

FIG. 2 is a flowchart of an illustrative process for imaging a fundus using an apparatus that includes a digital micromirror device (DMD) component, according to some embodiments.

FIG. 3 shows an example of a target imaging portion, according to some embodiments.

FIG. 4 shows example illumination patterns, according to some embodiments.

FIG. 5 shows an example of a sequence of images used to obtain a combined image, according to some embodiments.

FIG. 6 shows an example of a sequence of images used to obtain a combined image, according to some embodiments.

FIG. 7 shows an example of a sequence of images used to obtain a combined image, according to some embodiments.

FIG. 8 is a schematic diagram of an illustrative computing device with which aspects described herein may be implemented.

FIG. 9 is a flowchart of an illustrative process for color balancing, according to some embodiments of the technology described herein.

FIG. 10 is a flowchart of an illustrative process for capturing multiple images of a subject's eye during an imaging period, according to some embodiments of the technology described herein.

FIG. 11 is a flowchart of an illustrative process for capturing a fundus image using a rolling shutter, according to some embodiments of the technology described herein.

FIG. 12 is a flowchart of an illustrative process for using a DMD to project a fixation target onto a subject's fundus, according to some embodiments of the technology described herein.

### DETAILED DESCRIPTION

Aspects of the present disclosure provide for improved techniques to assist in imaging a target (e.g., an eye) that are suitable for use in an imaging apparatus operated by a user (e.g., the subject, a clinician, a technician, a doctor, etc.). In some embodiments, the imaging apparatus includes one or more imaging devices including at least a fundus imaging device and a digital micromirror device (DMD).

Devices for fundus imaging require precise positioning of the eye and the device with respect to one another. For example, the pupil should be positioned with respect to the imaging device such that it is substantially centered and correctly spaced along the planned beam path of the imaging device. When properly aligned, light is transmitted along a beam path through the pupil, where it is projected onto the fundus. When improperly aligned, portions of the eye other than the pupil (e.g., the cornea) are illuminated. Illumination outside of the pupil is not useful. Rather, it causes unnecessary exposure and stray reflections, resulting artifacts that decrease image quality and make the image unsuitable for clinical use.

However, even when properly aligned, conventional illumination techniques are limited by the size of the pupil, which generally varies between patients. In particular, conventional illumination techniques, such as ring illumination and crescent shape side illumination, are limited by how the paths for illumination and imaging are separated on the pupil and the cornea. Due to these limitations, conventional illumination techniques cannot be implemented in patients with pupils having a diameter smaller than around three millimeters.

Techniques have been suggested for reducing the negative effects of imaging small pupils using the conventional illumination techniques. For example, polarization, anti-reflective coatings, and light baffling methods have been used to reduce stray light and reflections off of the cornea and optical elements. However, all of these methods are fixed and prove to be useful only in the special cases for which they are optimized.

In addition to the challenges associated with properly aligning the illumination path and imaging small pupils, the inventors have recognized other challenges associated with conventional techniques for fundus imaging. One such challenge is that conventional fundus imaging systems typically utilize one or more fixed white light emitting diodes (LEDs) for illuminating the fundus. The use of fixed white LEDs limits the ability to optimize properties of resulting images, such as color balance. Furthermore, the use of fixed white LEDs results in a bright flash, which is harsh on the eyes and causes quick pupil constriction (e.g., on the order of 10-100 milliseconds). As such, conventional imaging systems are typically limited to capturing a single fundus image after illumination, with the time between consecutive images depending on the time it takes for the pupil to re-dilate. As such, this limits the potential benefits associated with capturing multiple images back-to-back.

Accordingly, the inventors have developed techniques that address the above-described challenges associated with the conventional techniques. In some embodiments, the techniques include imaging the fundus using an imaging apparatus that includes a digital micromirror device (DMD). By using a DMD, optimized illumination patterns can be dynamically adapted for each patient, where any misalignment or ocular movements can also be compensated for in real time. For example, in some embodiments, the techniques include projecting a first illumination pattern onto the subject's eye using the DMD, and capturing an image of the pupil after the first illumination pattern is projected onto the subject's eye. To avoid causing the pupil to constrict prior to fundus imaging, the first illumination pattern may be projected using an IR illumination source. In some embodiments, the image of the pupil may be used to determine whether the first illumination pattern was projected onto non-target (e.g., non-pupil) portions of the subject's eye. If the first illumination pattern was projected onto non-target portions of the eye, the DMD may be used to tailor subsequent illumination patterns to avoid projection onto the non-target portions. If the first illumination pattern was not projected onto non-target portions of the eye, an image of the fundus may be captured. For example, the image of the fundus may be captured after a second illumination pattern is projected onto the eye using the DMD and a white light illumination source. The second illumination pattern may be constrained by the boundaries of the first illumination pattern to avoid projecting light onto non-target portions of the eye during fundus imaging.

In some embodiments, the techniques developed by the inventors include using a DMD in connection with a multispectral illumination source. For example, the illumination source may include multi-colored lasers and/or LEDs. As such, the DMD may be used in conjunction with the illumination source to spatially adjust the color of light used to illuminate the fundus, thereby enabling dynamic color balancing while imaging the fundus.

In some embodiments, the techniques developed by the inventors include using a DMD to project a sequence of illumination patterns onto the fundus to capture multiple fundus images before the pupil constricts. This has several benefits. For example, the multiple fundus images can be used to generate a single high-resolution image. Additionally or alternatively, the DMD can be used to project a sequence of illumination patterns in coordination with a rolling shutter of the imaging device, thereby enabling, among other benefits and applications, the reduction of motion artifacts that often result when the entire fundus is illuminated using a white LED. Additionally or alternatively, the DMD can be used to project a sequence of low-brightness illumination patterns for capturing images over longer durations (e.g., on the order of 20-30 ms), which is gentler on the patient's eye.

Following below are descriptions of various concepts related to, and embodiments of, techniques for imaging the fundus using an apparatus comprising a digital micromirror device (DMD). It should be appreciated that various aspects described herein may be implemented in any of numerous ways, as the techniques are not limited to any particular manner of implementation. Examples of details of implementations are provided herein solely for illustrative purposes. Furthermore, the techniques disclosed herein may be used individually or in any suitable combination, as aspects of the technology described herein are not limited to the use of any particular technique or combination of techniques.

### Exemplary Apparatus for Fundus Imaging

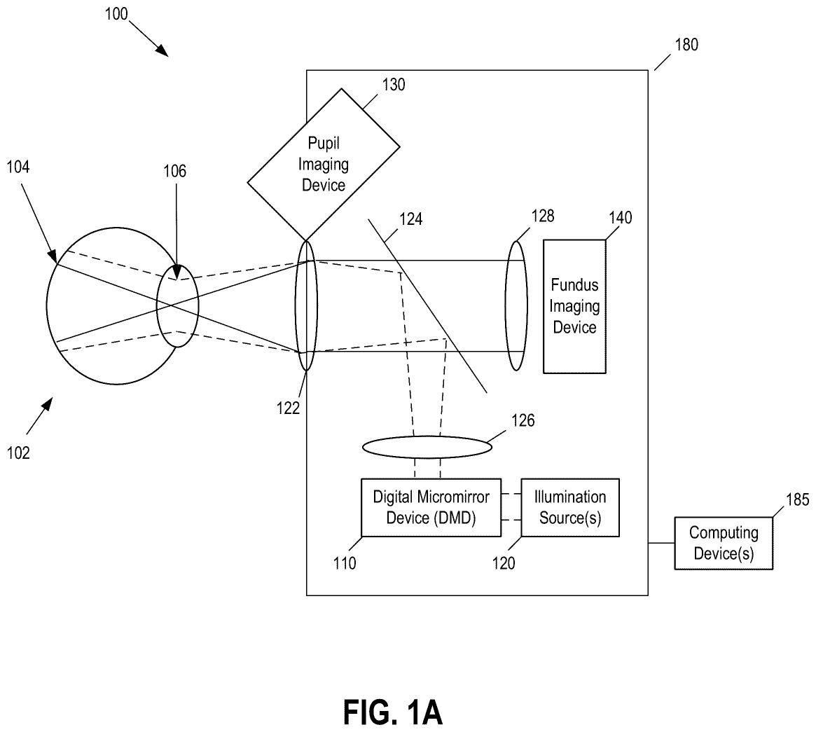

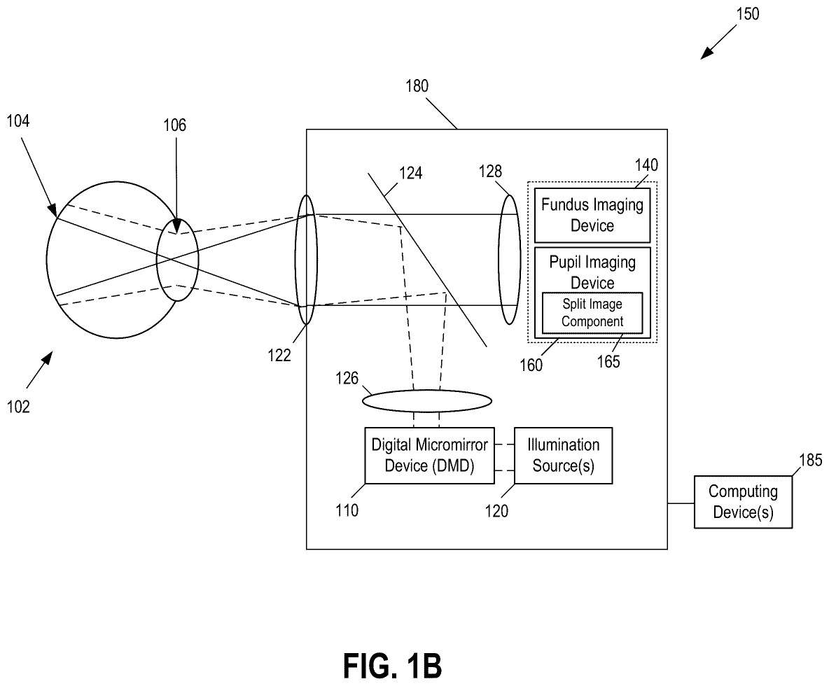

FIG. 1A is a schematic view of an example fundus imaging system, according to a first embodiment. As shown, system 100 includes pupil imaging device 130, fundus imaging device 140, a digital micromirror device (DMD) 110, illumination source(s) 120, objective lens 122, and computing device(s) 185. In some embodiments, the system 100 additionally, or alternatively, includes beam splitter 124, lens 126, and lens 128. It should be appreciated that system 100 include one or more additional, or alternative, components which are not illustrated in FIG. 1A. For example, system 100 may include computing device(s), actuator(s), additional lens(es), mirror(s), and/or additional beam splitter(s). In some embodiments, one or more (e.g., all) of the components of system 100 may be included in an imaging apparatus.

In some embodiments, the imaging system 100 may be configured to support an optical path between the DMD 110 and the subject's eye 102. In the example shown in FIG. 1A, the DMD 110 is configured to use illumination source(s) 120 to project illumination pattern(s) onto the retina plane 104. In particular, light is directed through lens 126, reflects off of beam splitter 124, and is transmitted through the objective lens 122, after which the illumination pattern is projected onto the retina plane 104. In the example of FIG. 1A, the pupil and apparatus are aligned, and the illumination pattern has been tailored to the pupil, such that the illumination pattern is projected through the pupil 106 only (as opposed to being projected onto the cornea, or other non-target portions of the eye) and onto the retina plane 104.

As described herein, a DMD is an array of individually switchable mirrors that can be used as a rapid spatial light modulator. DMD 110 may include any suitable number of DMDs, each of which may be of any suitable size and include any suitable number of mirrors, as aspects of the technology described herein are not limited in this respect. In some embodiments, DMD 110 may be integrated into a digital light processing (DLP) projector.

In some embodiments, DMD 110 may be used in conjunction with illumination source(s) 120. Illumination source(s) 120 may include any suitable source components such as, for example, light emitting diodes (LED), infrared (IR) light sources, lasers, transparent glass tubes filled with an inert gas (e.g., xenon gas or other noble gas), a quartz tube filled with an inert gas, and/or any other suitable components that are configured to generate illumination light, as aspects of the technology described herein are not limited in this respect. For example, the illumination source(s) 120 may include (a) one or more IR illuminators (e.g., one or more LEDs and/or lasers configured to emit IR light) and/or (b) a white light source (e.g., one or more LEDs). In some embodiments, when the illumination source(s) 120 include a white light source, the white light source may include a high brightness LED. Additionally or alternatively, the illumination source(s) 120 may include one or more colored LEDs. For example, the colored LEDs may include a red LED, a greed LED, a blue LED, and/or LED(s) of any other suitable color(s), as aspects of the technology described herein are not limited in this respect. Additionally or alternatively, the illumination source(s) 120 may include one or more colored lasers. For example, the colored lasers may include a red laser, a greed laser, a blue laser, and/or laser(s) of any other suitable color(s), as aspects of the technology described herein are not limited in this respect.

In some embodiments, the imaging system 100 may further be configured to support an optical path between the fundus imaging device 140 and the subject's eye 102. In the example shown in FIG. 1A, fundus imaging device 140 is configured receive light that has been reflected off of the subject's eye 102 and has been transmitted through both the objective lens 122 and beam splitter 124. In some embodiments, the fundus imaging device 140 is configured to capture an image of the fundus using the received light.

In some embodiments, the fundus imaging device 140 may include one or a plurality of image sensors. Such an image sensor may include any suitable image sensor configured to use the received light to generate an image, as aspects of the technology described herein are not limited in this respect. In some embodiments, the image sensor includes a sensing element such as, for example, a two-dimensional array of pixels. The sensing element may be monochrome or color. The sensing element may be a complementary metal-oxide-semiconductor (CMOS) chip, charge-coupled device (CCD) chip, or any other suitable sensing element, as aspects of the technology described herein are not limited in this respect.

In some embodiments, the image sensor(s) of the fundus imaging device 140 employ a rolling shutter. When an image sensor employs a rolling shutter, the sensor pixels are read out row-by-row (or column-by-column). In some embodiments, the image sensor(s) of the fundus imaging device 140 employ a global shutter. When an image sensor employs a global shutter, the sensor pixels are read out substantially simultaneously.

In some embodiments, the fundus imaging system 100 implements confocal gating, which may help to extend imaging depth. For example, the fundus imaging system 100 may include one or more components through which light (e.g., light reflected from the eye 102) may pass before it arrives at the fundus imaging device 140. For example, in some embodiments, though not shown, the DMD 110 may be positioned along an optical path (e.g., an imaging path) between the subject's eye 102 and the fundus imaging device 140. For example, the fundus imaging device 140 may receive light that has reflected off the subject's eye 102 and passed back through the DMD 110, thereby resulting in confocal gating.

In some embodiments, the imaging system 100 may further be configured to support an optical path between the pupil imaging device 130 and the subject's eye. In some embodiments, the pupil imaging device 130 is configured to capture an image of the pupil using light reflected from the subject's eye.

In some embodiments, the pupil imaging device 130 may include one or a plurality of image sensors. Such an image sensor may include any suitable image sensor configured to use the received light to generate an image, as aspects of the technology described herein are not limited in this respect. In some embodiments, the image sensor includes a sensing element such as, for example, a two-dimensional array of pixels. The sensing element may be monochrome or color. The sensing element may be a complementary metal-oxide-semiconductor (CMOS) chip, charge-coupled device (CCD) chip, or any other suitable sensing element, as aspects of the technology described herein are not limited in this respect.

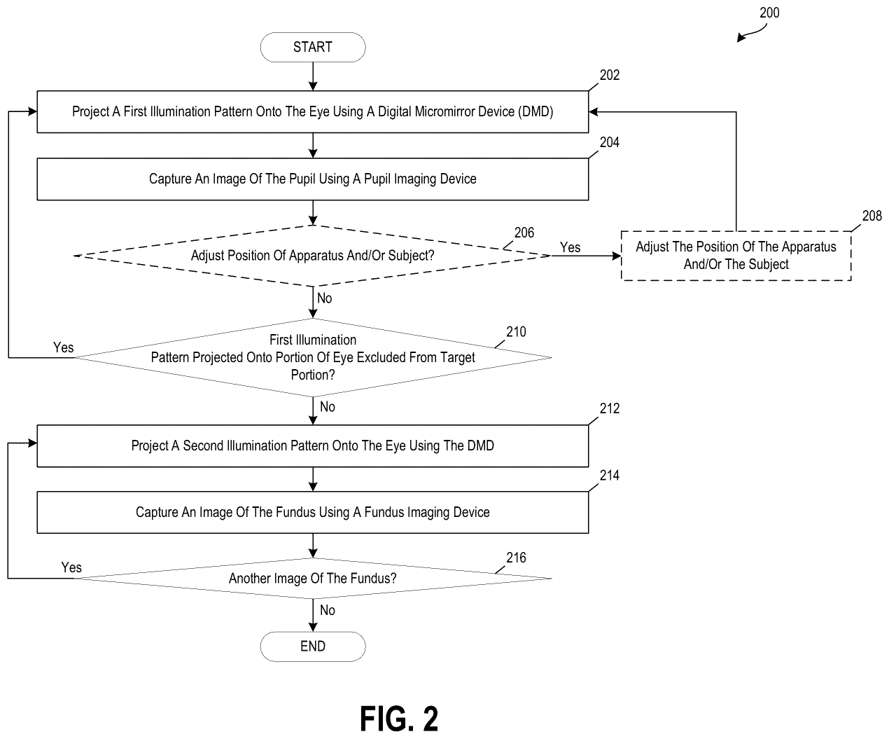

In some embodiments, the pupil imaging device 130 includes multiple stereo image sensors that can be configured to generate and/or output analog and/or digital data representative of a stereo image. In some embodiments, the stereo images. As described herein including at least with respect to FIG. 2, such a stereo image may be used to align an imaging path of system 100 with the pupil. Though not shown, system 100 may include one or more actuator(s) configured to assist in the alignment by automatically adjusting the position of one or more components of system 100.

As shown in FIG. 1A, the optical path between the pupil imaging device 130 and the subject's eye 102 is distinct from the optical path between the fundus imaging device 140 and the subject's eye 102. However, in alternative embodiments it can be in the same optical path of the fundus imaging device 140.

In some embodiments, imaging system 100 additionally includes one or more computing device(s) 185. For example, the computing device(s) 185 may be used to control components of the imaging system such as, for example, the DMD 110, the illumination source(s) 120, the fundus imaging device 140, and/or the pupil imaging device 130. Additionally, or alternatively, the computing device(s) may be configured to perform one or more acts of one or more processes, such as process 200 shown in FIG. 2. It should be appreciated that while the computing device(s) 185 are illustrated as being external to apparatus 180, the computing device(s) 185 may be included within apparatus 180.

FIG. 1B shows an alternative embodiment of an example imaging system. Imaging system 150 shown in FIG. 1B may be the same as imaging system 100 except that imaging system 150 includes pupil imaging device 160. Pupil imaging device 160 may be the same as pupil imaging device 130 except that pupil imaging device 160 shares at least a portion of an optical path with fundus imaging device 140. For example, the optical path between the subject's eye 102 and pupil imaging device 160 may include at least the objective lens 122. Though illustrated together, it should be appreciated that the optical path of the fundus imaging device 140 may diverge from the optical path of the pupil imaging device 160 via one or more optical components (not shown) such as, for example, one or more beam splitters, lenses, mirrors, or any other suitable optical component(s).

In some embodiments, the pupil imaging device 160 additionally, or alternatively, includes a split image component 165 such as, for example, a split prism or a split mirror. In some embodiments, the split image component 165 is configured to generate a split image, and the split image may be used to align the pupil with the imaging path of imaging system 150. Techniques for performing such an alignment are described herein including at least with respect to FIG. 2. As described above, one or more actuator(s) (not shown) of system 150 may be used to assist in the alignment.

### Exemplary Fundus Imaging Techniques—Pupil Alignment

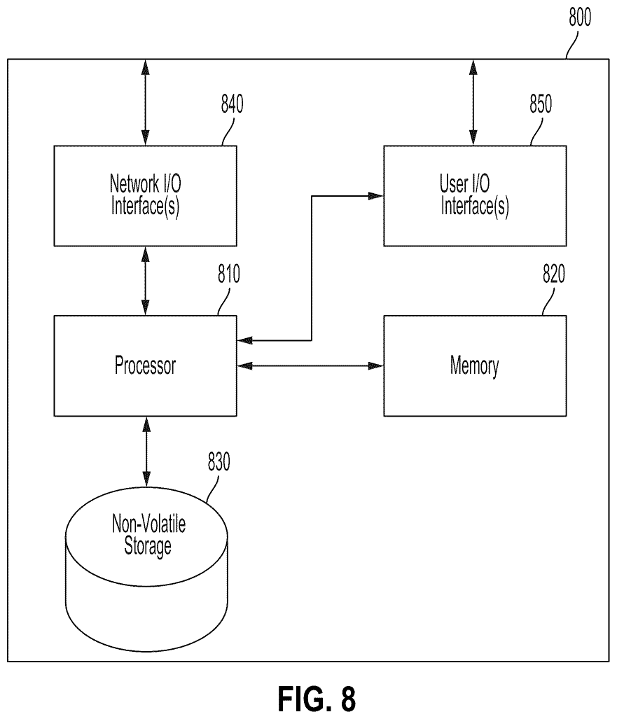

FIG. 2 is a flowchart of an illustrative process for imaging a subject's fundus, according to some embodiments. One or more of acts (e.g., all of the acts) of process 200 may be performed automatically by any suitable computing device(s). For example, the act(s) may be performed by a System on Module (SOM) computer, laptop computer, a desktop computer, one or more servers, in a cloud computing environment, computing device 800 described herein with respect to FIG. 8, and/or in any other suitable way.

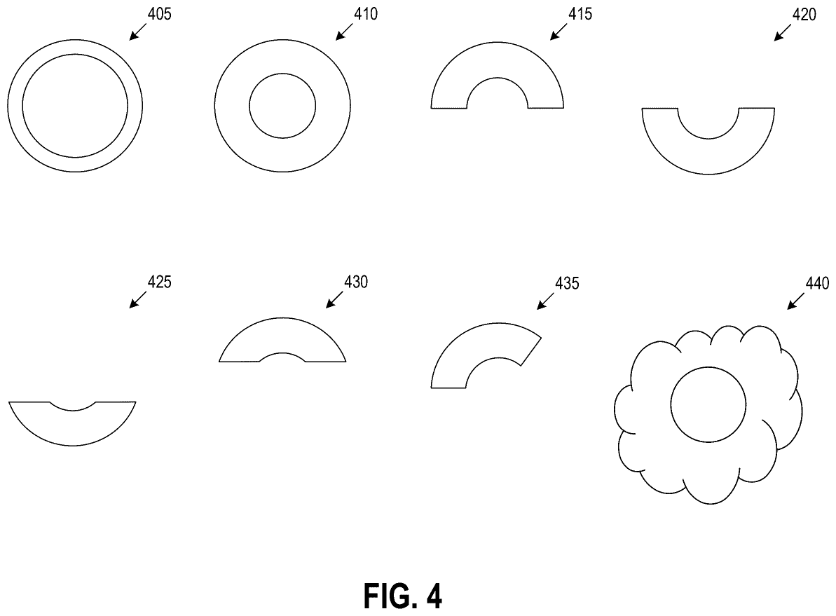

At act 202, a first illumination pattern is projected onto the eye using a digital micromirror device (DMD) (e.g., the DMD 110 shown in FIGS. 1A and 1B). The first illumination pattern may include any suitable illumination pattern, as aspects of the technology are not limited in this respect. Nonlimiting examples of illumination patterns are shown in FIG. 4 (e.g., example pattern 405, pattern 410, pattern 415, pattern 420, pattern 425, pattern 430, pattern 435, and pattern 440).

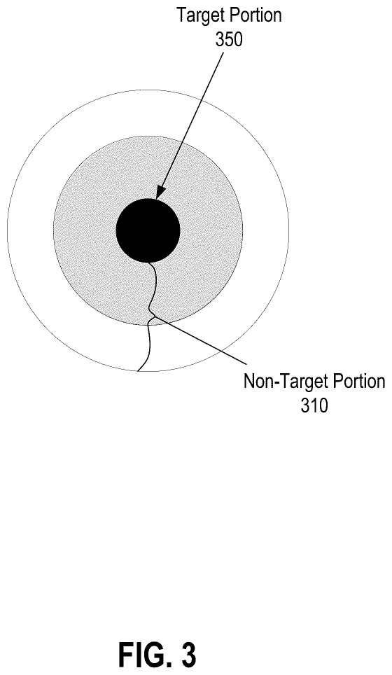

In some embodiments, the DMD is configured to project an illumination pattern having particular dimensions. The dimensions may be determined using any suitable techniques, as aspects of the technology described herein are not limited in this respect. For example, the dimensions may be determined based on an estimated or measured size of a target portion of the eye (e.g., the pupil). In particular, the dimensions may be determined in an effort to project the illumination pattern onto the target portion of the eye (e.g., the pupil), rather than onto a non-target portion of the eye (e.g., the cornea). FIG. 3 shows an example of a target 350 and non-target 310 portion of the eye.

When the dimensions of the first illumination pattern are determined based on the size of the pupil, the size of the pupil may be estimated or measured using any suitable techniques, as aspects of the technology described herein are not limited in this respect. For example, the size of the pupil may be estimated based on pupil sizes measured for one or more other subjects (e.g., an average pupil size). Additionally, or alternatively, the size of the pupil of the particular subject may be measured using any suitable pupil measurement techniques, as aspects of the technology described herein are not limited in this respect. For example, the size of the subject's pupil may be measured based on a previously captured image of the subject's pupil. In this case, the size of the pupil may be measured by (a) measuring the size (e.g., the diameter) of the pupil in the image, and (b) multiplying the measured size by a scaling value. In some embodiments, the image is captured using the pupil imaging device described with respect to act 204.

In some embodiments, the DMD projects the first illumination pattern onto the eye using an illumination source. To avoid causing the pupil to constrict in size prior to imaging the fundus, the illumination source may include an infrared illumination source. Alternatively, the illumination source may include any other suitable illumination source such as, for example, a white light illumination source, as aspects of the technology described herein are not limited in this respect.

At act 204, an image of the pupil is captured using a pupil imaging device. In some embodiments, the image is captured after the first illumination pattern is projected onto the eye. An example of the pupil imaging device is described herein including at least with respect FIG. 1A and FIG. 1B.

At act 206, it is determined whether to adjust a position of the apparatus (e.g., the apparatus comprising the DMD) and/or a position of the subject based on the image captured at act 206. In some embodiments, this includes determining whether the fundus imaging device is aligned with the pupil. This may be achieved using any suitable techniques, as aspects of the technology described herein are not limited in this respect. If the fundus imaging device is aligned with the pupil, then no adjustment may be needed. If the fundus imaging device is not aligned with the pupil, then an adjustment may be needed.

One example technique for determining whether the fundus imaging device is aligned with the pupil includes localizing a pupil in a field of view of the fundus imaging device using a split image of the fundus. For example, as described herein including at least with respect to FIG. 1B, the pupil imaging device may include a split image component (e.g., a split prism or a split mirror), and the image captured at act 204 may be a split image of the pupil. The split image may be split into image portions (e.g., halves, quarters, etc.). If the portions of the split image are aligned with one another, and the pupil is centered in the image, this may indicate that the fundus imaging device is aligned with the pupil. If the portions of the split image are misaligned, this may indicate that the pupil is positioned too close or too far from the fundus camera. The degree of displacement between the misaligned image portions may be used to determine how to position the subject and/or the fundus imaging device such that they are aligned. If the pupil is not centered in the image, this may indicate that the fundus imaging device and pupil are misaligned along a plane perpendicular to the imaging path. The displacement between the center of the pupil and the center of the image may be used to determine how to position the subject and/or the fundus imaging device such that they are aligned.

As an additional, or alternative example, determining whether the fundus imaging device is aligned with the pupil may include determining a gaze angle of the subject's eye. In some embodiments, the gaze angle is determined based on a stereo image of the subject's eye. For example, the pupil imaging device may include a stereo camera, and the image captured at act 204 may be a stereo image. Alternatively, the image captured at act 204 may be one of two images used to generate a stereo image. In some embodiments, determining the gaze angle of the subject's eye includes using the stereo image to determine the major and minor axes of the pupil, and determining the gaze angle based on the major and minor axes. If the gaze angle is oriented towards an intended target (e.g., a fixation target), this may indicate that the pupil is aligned with the fundus imaging device. If the gaze angle is not oriented towards the intended target, then this may indicate that the pupil is not aligned with the fundus imaging device, and the measured gaze angle may be used to determine how to position the fundus imaging device such that they are aligned. Example techniques for determining gaze angle are described in U.S. Provisional Patent Application No. 63/588,609, which is incorporated by reference herein in its entirety.

If, at act 206, it is determined that a position of the apparatus and/or subject should be adjusted, process 200 proceeds to act 208. At act 208, a position of the apparatus and/or subject is adjusted. In some embodiments, adjusting the position of the subject includes outputting instructions to an operator of the apparatus (e.g., the subject or another user) with respect to how the subject should be repositioned. In some embodiments, adjusting the position of the apparatus includes automatically adjusting the position of the apparatus using an actuator configured to adjust the position of the apparatus. Alternatively, adjusting the position of the apparatus includes outputting instructions to an operator of the apparatus (e.g., the subject or another user).

After the position of the subject and/or apparatus is adjusted at act 208, process 200 returns to act 202. For example, acts 202, 204, 206, and 208 may be repeated until it is determined, at act 206, that the position of the subject and/or apparatus should not be adjusted.

If, at act 206, it is determined that the position of the apparatus and/or subject should not be adjusted, process 200 proceeds to act 210. At act 210, it is determined whether the first illumination pattern was projected onto a portion of the eye that is excluded from a target portion. In some embodiments, this may include evaluating otherwise processing the image captured at 204 to determine whether the first illumination pattern was projected onto a portion of the eye excluded from the target region. This may include user evaluation of the image. For example, the user may visually inspect the image. If the first illumination pattern was projected onto a non-target portion of the eye, then the image may include reflections and/or other image artifacts. Alternatively, any suitable image processing techniques may be used to determine whether the first illumination pattern was projected onto a non-target portion of the eye. For example, the image may be processed using a machine learning model trained to predict whether the illumination pattern was projected onto non-target portions of the eye and/or identify portions of the eye that have been illuminated.

If, at act 210, it is determined that the first illumination pattern was projected onto a portion of the eye excluded from the target portion, process 200 returns to act 202, during which an illumination pattern different from the first illumination pattern is projected onto the eye using the DMD. In some embodiments, the illumination pattern is generated based on the image that was captured at act 204. For example, the position of the artifacts and/or reflections in the image may be used, as feedback, to determine how to adjust the illumination pattern (e.g., using the DMD) to avoid again projecting the illumination pattern onto the non-target portions of the eye. Additionally, or alternatively, the image captured at act 204 may be used to determine updated measurements of the pupil (e.g., as described with respect to act 202), and the illumination pattern may be tailored to the updated measurements.

After the illumination pattern is generated and projected onto the subject's eye at act 202, one or more of acts 204, 206, 208, and 210 may be repeated.

If, at act 210, it is determined that the first illumination pattern was not projected onto a portion of the eye excluded from the target portion (e.g., the illumination pattern was projected solely onto the subject's pupil), process 200 proceeds to act 212. At act 212, a second illumination pattern is projected onto the eye using the DMD. In some embodiments, the second illumination pattern is the same illumination pattern (e.g., same size and shape) as the first illumination pattern. Alternatively, the second illumination pattern may be different from the first illumination pattern. When the second illumination pattern is different from the first illumination pattern, the second illumination pattern may be constrained by the boundaries of the first illumination pattern. For example, the boundaries of the second illumination pattern may be the same as the boundaries of the first illumination pattern or positioned within the boundaries of the first illumination pattern to avoid illuminating non-target portions of the subject's eye.

In some embodiments, the DMD projects the second illumination pattern onto the eye using an illumination source suitable for capturing a fundus image. For example, the illumination source may be a white light source.

At act 214, an image of the fundus is captured using a fundus imaging device. In some embodiments, the image is captured after the second illumination pattern is projected onto the eye. An example of the fundus imaging device is described herein including at least with respect FIG. 1A and FIG. 1B.

At act 216, it is determined whether another image of the fundus should be captured. For example, in some embodiments, it may be desirable to project different illumination patterns onto the eye and capture a respective sequence of images. For example, with reference to FIG. 4, one fundus image may be captured after example illumination pattern 415 is projected onto the subject's eye, and a subsequent image may be captured after example illumination pattern 420 is projected onto the subject's eye. Due to the DMD's ability to rapidly modify the illumination pattern, such a sequence of images may be captured within a very short time of one another before the pupil constricts (e.g., within milliseconds of one another). Any suitable number of images may be captured in the sequence of images within the time constraint imposed by pupil constriction, as aspects of the technology are not limited in this respect.

Accordingly, if, at act 216, it is determined that another image should be captured, act 212 and act 214 are repeated to capture another fundus image in the sequence of fundus images. As described above, at act 212, an illumination pattern different from the second illumination pattern may be projected onto the eye. At act 214, the respective fundus image may be captured.

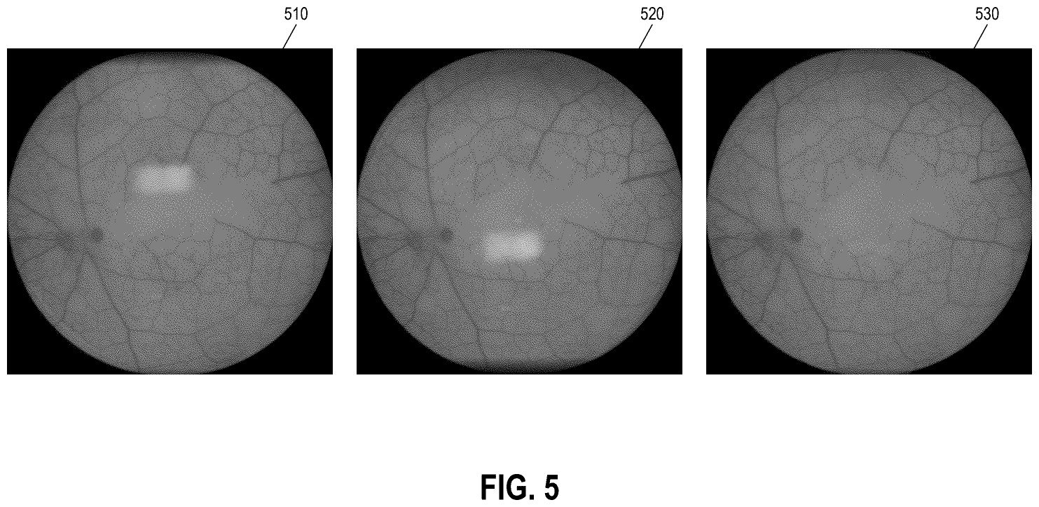

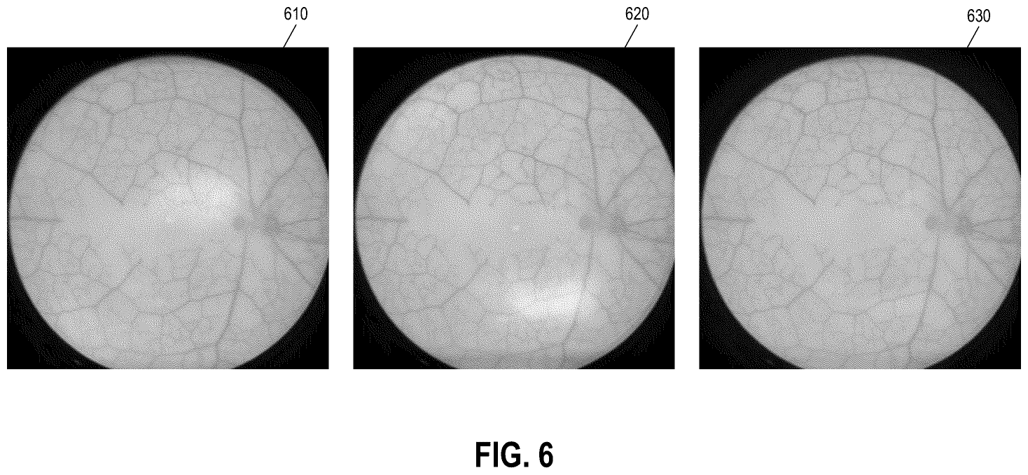

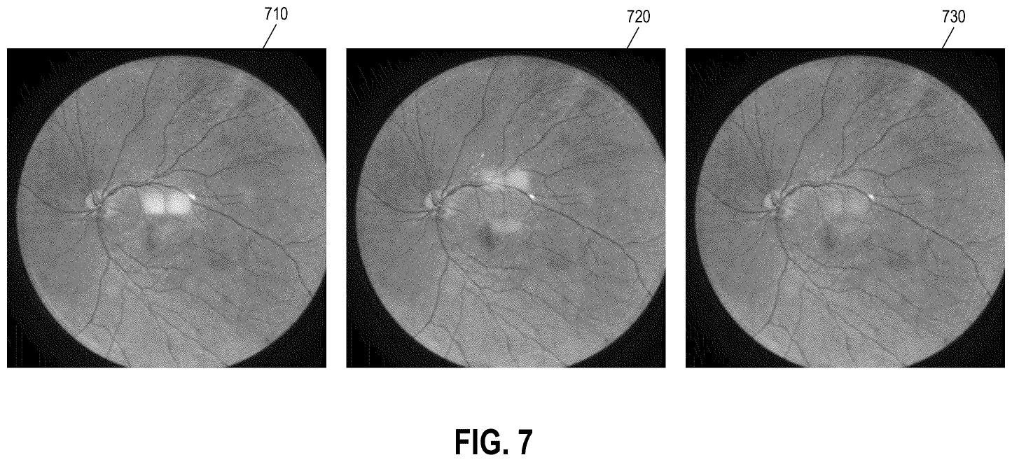

In some embodiments, if a sequence of fundus images is captured during acts 212-216, said images may be combined to generate one or more combined fundus images. Combining the fundus images may improve the contrast, eliminate artifacts (e.g., reflections), and/or improve one or more other characteristics of the resulting combined fundus image(s). FIG. 5, FIG. 6, and FIG. 7 each show an example of two fundus images that were combined to generate a third, combined image that excludes artifacts depicted in the two individual images. In particular, with respect to FIG. 5, image 510 and image 520 were combined to generate image 530. With respect to FIG. 6, image 610 and image 620 were combined to generate image 630. With respect to FIG. 7, image 710 and image 720 were combined to generate image 730.

If it is determined that another image should not be captured, process 200 ends.

### Exemplary Fundus Imaging Techniques—Color Balancing

In some embodiments, the techniques developed by the inventors can be used to perform spatial color balancing to obtain color balanced fundus images. Spatial color balancing can allow for higher color contrast in portions of an image corresponding to specific areas of the eye, or at the position of specific pathologies. In some embodiments, spatial color balancing is performed by controlling exposure. For example, exposure can be controlled using imaging components such as a multispectral illumination source, a DMD and/or an image sensor.

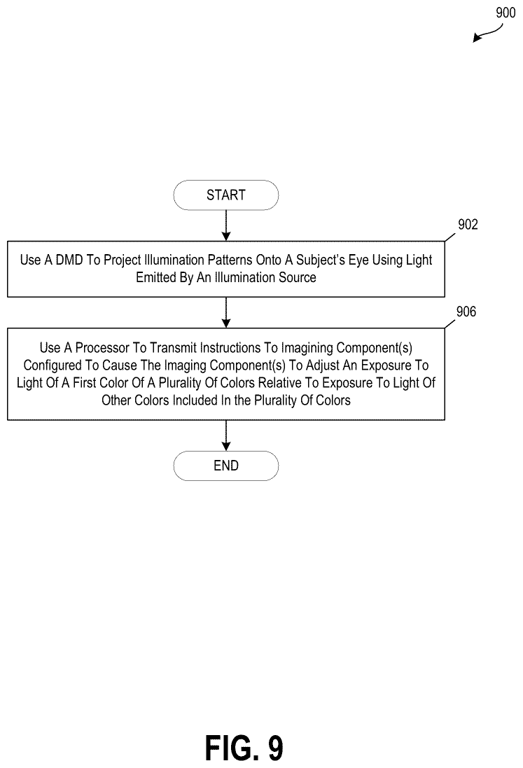

FIG. 9 is a flowchart of an illustrative process 900 for multispectral imaging, according to some embodiments of the technology described herein. In some embodiments, process 900 is performed using a processor and one or more imaging components. For example, the imaging components may include a DMD, an illumination source, and at least one image sensor.

At act 902, one or more of the imaging components are used to illuminate at least a portion of the subject's eye. For example, a DMD may be used to project one or more illumination patterns onto the subject's eye using light emitted by an illumination source. In some embodiments, the illumination source is configured to emit light of a plurality of colors. The illumination source may comprise a multispectral illumination source. For example, the illumination source may include one or multiple color LEDs. For example, the illumination source may include one or more red LEDs, one or more blue LEDs, one or more green LEDs, and/or one or more LEDs of any other suitable color. Additionally or alternatively, the illumination source may include one or multiple colored lasers. For example, the illumination source may include one or more red lasers, one or more blue lasers, one or more green lasers, and/or one or more lasers of any other suitable color. The illumination source may include illumination source 120 shown in FIG. 1A and FIG. 1B. The DMD may include the DMD 110 shown in FIG. 1A and FIG. 1B.

At act 904, a processor is used to transmit instructions to one or more of the imaging components. In some embodiments, the instructions are configured to cause the one or more imaging components to adjust an exposure of a resulting image captured using the one or more imaging components. For example, the instructions may be configured to cause the one or more imaging components to adjust the exposure to light of a first color relative to other colors.

In some embodiments, the image sensor is used to adjust the exposure to light of the first color. For example, adjusting the exposure may include adjusting the conversion gain associated with one or more pixels of an image sensor used to capture the image. For example, increasing the gain may increase the exposure, while decreasing the gain may decrease the exposure. The exposure may be adjusted spatially by adjusting the gain of a pixel or subset(s) of pixels of the image sensor. The exposure may be adjusted temporally by adjusting the gain over time. For example, the gain of one or more pixels may be adjusted when light of a first color is projected onto the eye, and then readjusted when light of a second color is projected onto the eye.

Additionally or alternatively, the illumination source may be used to adjust the exposure. For example, the intensity of the illumination source may be adjusted to adjust the exposure; increasing the intensity of the illumination source may increase the exposure, while decreasing the intensity of the illumination source may decrease the exposure. The intensity of the illumination source may be adjusted spatially and/or temporally. For example, light of the same color or different colors may be projected at different intensities onto different regions of the eye. Additionally or alternatively, the intensity of the light may be adjusted over time.

Additionally or alternatively, the DMD and the imaging sensor may be used to adjust the exposure. For example, the exposure may be adjusted by adjusting the rate at which the DMD projects illumination patterns onto the subject's eye and the corresponding shutter speed of the image sensor. For example, increasing the rate of light projection and shutter speed may decrease exposure, while decreasing rate of light projection and shutter speed may increase the exposure. The shutter speed and illumination projection rate may be adjusted as light of different colors is projected onto the eye.

In some embodiments, the exposure adjustment is global. For example, the exposure to light of the first color may be adjusted across the entire resulting image. In some embodiments, the exposure adjustment is local. For example, the exposure to light of the first color may be adjusted in certain portions of the image. For example, the color intensity may be adjusted in regions corresponding to one or more anatomical and/or pathological features.

### Exemplary Fundus Imaging Techniques—Multiple Image Capture

As described herein, the DMD can be used to dynamically adjust illumination patterns projected onto the subject's eye. Capturing multiple images using different illumination patterns has many potential advantages and applications.

### High Resolution Imaging

In some embodiments, a fundus imaging system that includes a DMD can be used to generate high-resolution images without repetitively using a bright white flash.

As described herein, conventional fundus imaging techniques involve illuminating the eye using a white light illumination source, then capturing a single fundus image before the pupil constricts. The period between the initial illumination and a time point prior to pupil constriction (also referred to as an “imaging period”) typically ranges from 10 to 100 milliseconds. Oftentimes, multiple images are captured due to artifacts that are present in previously captured image. As a result, the imaging process is time consuming and harsh on the subject's eye.

However, by employing a DMD, the techniques developed by the inventor enable the projection of several different illumination patterns onto the subject's fundus during a single imaging period. As such, in some embodiments, the techniques developed by the inventors involve projecting multiple illumination patterns onto the subject's fundus during an imaging period and, for each illumination pattern, capturing a respective image of the subject's fundus, thereby obtaining a plurality of fundus images.

In some embodiments, the plurality of fundus images can be combined to generate a single fundus image. Techniques for combining images are described herein including at least with respect to act 214 of process 200 shown in FIG. 2. In some embodiments, the images are combined using a super-resolution technique.

In some embodiments, the resulting, combined image has a higher resolution relative to the resolution of the individual fundus images.

### Video Illumination

In some embodiments, a fundus imaging system that includes a DMD can be used to project low-brightness video sequences onto the subject's eye to capture images, which is gentler on the subject's eye. A video sequence may refer to a sequence of illumination patterns that are consecutively projected onto the subject's eye. For example, the video sequence may be a movie that is projected (e.g., continuously projected) onto the subject's eye during an imaging period. One or more imaging components may be used to dim the brightness of the video sequence. For example, the DMD and/or the illumination source may be used to dim the brightness of the illumination patterns projected onto the eye. Using a lower brightness prevents the pupil from rapidly constricting, as compared to when using a bright light source (e.g., where the pupil constricts on the order of 10-100 milliseconds).

As such, in some embodiments, the DMD is used to project a low-brightness video sequence onto the eye during a relatively long imaging period. For example, the DMD may be used to project the low-brightness video sequence onto the eye for between 20-30 seconds. The brightness (or intensity) may be less than or equal to a threshold brightness (or intensity). In some embodiments, the DMD continues to project the video sequence onto the subject's eye during one or more blinks of the subject's eye.

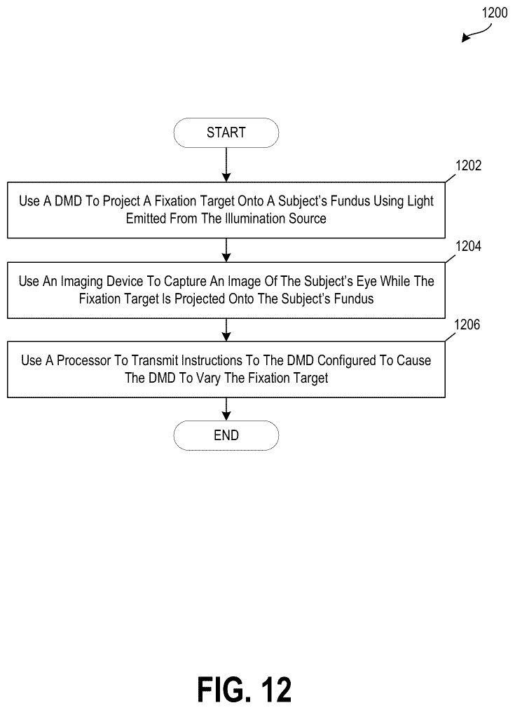

In some embodiments, the illumination patterns may additionally or alternatively integrate aspects of subject feedback. For example, the illumination patterns may include a fixation target indicating where the subject should look or how the subject should move his or her eye. Examples of fixation targets are described herein including at least with respect to FIG. 12.

In some embodiments, while the DMD is used to project the low-brightness illumination patterns, a fundus imaging device is used to capture images during the imaging period. In some embodiments, the images are captured in between blinks of the subject's eye.

The captured images may be combined to generate a single combined image. For example, combining the images may involve co-registering and integrating the images to generate a single image. Combining multiple images to generate a single image may result in a combined image having reduced noise and/or increased resolution.

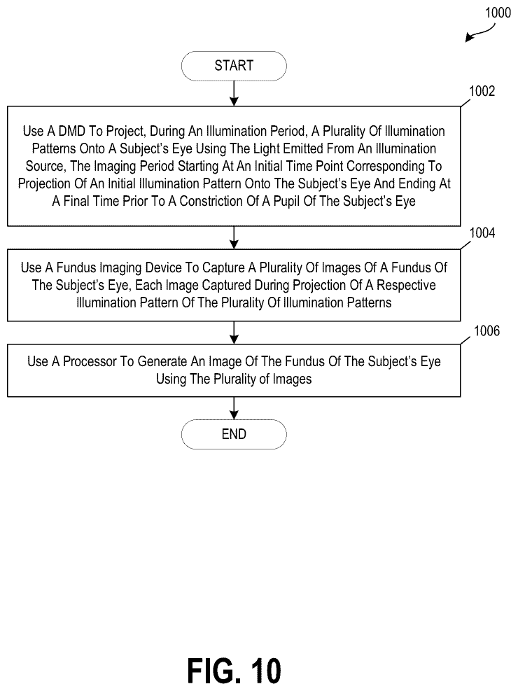

FIG. 10 is a flowchart of an illustrative process 1000 for capturing multiple images of a subject's eye during an imaging period, according to some embodiments of the technology described herein.

At act 1002, a DMD is used to project a plurality of illumination patterns onto a subject's eye during an illumination period. In some embodiments, the illumination period starts at an initial time point corresponding to projection of an initial illumination pattern onto the subject's eye and ends at a final time point prior to or corresponding to constriction of the subject's eye. The DMD may include the DMD 110 described herein including at least with respect to FIG. 1A and FIG. 1B.

As described herein, the DMD may be used to project multiple illumination patterns using a bright white and/or multispectral illumination source. For example, the DMD may be used to project multiple illumination patterns using a bright white and/or multispectral illumination source during an imaging period of between 5 and 150 milliseconds.

Additionally or alternatively, the DMD may be used to dim the brightness of the illumination pattern projected onto the subject's eye. For example, the dimmed illumination pattern is a video sequence. The brightness (or intensity) may be less than or equal to a threshold brightness (or intensity). In some embodiments, the DMD is used to project the dimmed illumination patterns onto the subject's eye during an imaging period between 10 and 40 seconds.

At act 1004, a fundus imaging device is used to capture a plurality of images of a fundus of the subject's eye. In some embodiments, each image is captured during projection of a respective pattern of the plurality of illumination patterns. The fundus imaging device may include fundus imaging device 140 described herein including at least with respect to FIG. 1A and FIG. 1B.

At act 1006, a processor is used to generate an image of the fundus of the subject's eye using the plurality of images. The processor may include computing device(s) 185 descried herein including at least with respect to FIG. 1A and FIG. 1B.

In some embodiments, generating the image includes combining the multiple captured images. For example, the multiple captured images may be combined to include a single image that has a higher resolution than the individual captured images. Techniques for combining images are described herein including at least with respect to act 214 of process 200 shown in FIG. 2. In some embodiments, the images are combined using a super-resolution technique

Exemplary Fundus Imaging Techniques—Synchronization with a Rolling Shutter

As described herein, in some embodiments, a fundus imaging device employs a rolling shutter. When an image sensor employs a rolling shutter, the sensor pixels are read out row-by-row (or column-by-column). In some embodiments, the DMD is used to project illumination patterns in coordination with the rolling shutter.

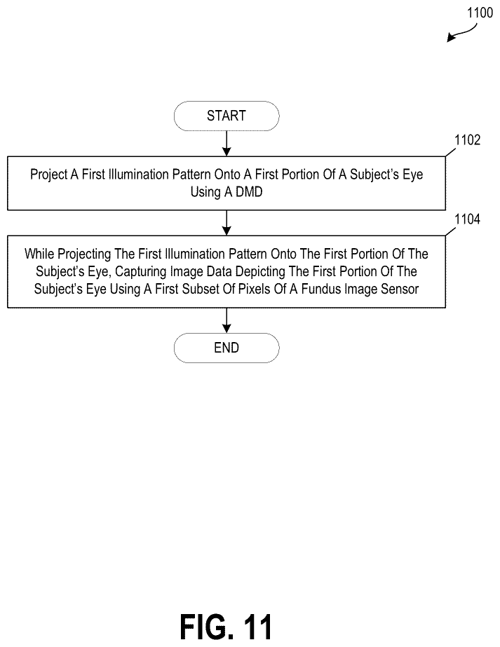

FIG. 11 is a flowchart of an illustrative process 1100 for capturing a fundus image using a rolling shutter, according to some embodiments of the technology described herein.

At act 1102, a first illumination pattern is projected on a first portion of a subject's eye using a DMD.

In some embodiments, the first illumination pattern is configured to illuminate a portion of the subject's eye that is depicted in the image data captured by the fundus image sensor at act 1104. In other words, in such embodiments, the DMD (and the illumination light emitted from the DMD) is synchronized with the image sensor's rolling shutter; the DMD is configured to illuminate the portion of the eye that is being imaged. Synchronizing the illumination with the rolling shutter can help to reduce motion blur by reducing exposure time.

At act 1104, while the DMD is used to project the first illumination pattern onto the subject's eye, a first subset of pixels of a fundus image sensor is used to capture image data depicting the portion of the subject's eye. In some embodiments, the first subset of pixels is aligned along a row or column (e.g., an integration line) of an array of pixels of the fundus image sensor.

In some embodiments, process 1100 may be repeated as the rolling shutter progresses to capture an image using the rest of the pixels in the image sensor. For example, the DMD may be used to adjust the position of the illumination pattern together with (e.g., synchronized with) the rolling shutter integration line.

### Exemplary Fundus Imaging Techniques—Fixation Target

In some embodiments, a fundus imaging system comprising a DMD is used to project illumination patterns onto the subject's eye that operate as fixation targets. A fixation target may be used to provide feedback to the subject. For example, a fixation target may be used to direct the subject where, when, and how to look or move his or her eye. A DMD may be used to dynamically change a fixation target.

FIG. 12 is a flowchart of an illustrative process 1200 for using a DMD to project a fixation target onto a subject's fundus, according to some embodiments of the technology described herein.

At act 1202, a DMD is used to project a fixation target onto a subject's fundus using light emitted from an illumination source. In some embodiments, the fixation target is an illumination pattern comprising one or more dots. In some embodiments, the fixation target comprises an illumination pattern that depicts a focus scene. In some embodiments, the fixation target includes written instructions for the subject. The fixation target may be used to instruct the subject when to look or move, where to look, and/or how to move. The fixation target may be used to draw the attention of the subject and/or to dilate the subject's pupil. In some embodiments, the fixation target is a dynamic gaze track pattern for the subject to follow with his or her eye.

In some embodiments, when projecting a fixation target onto the subject's eye, the DMD is used to dim the brightness of the light from the illumination source, thereby limiting constriction of the pupil. For example, the DMD may be configured to project a low-brightness video sequence onto the subject's eye, as described herein including at least with respect to FIG. 10.

At act 1204, an imaging device is used to capture an image of the subject's eye while the fixation target is projected onto the subject's fundus. For example, the imaging device may include a pupil imaging device configured to capture images and/or video of the subject's pupil as it follows the fixation target (e.g., as it follows a dynamic gaze track pattern.) In some embodiments, capturing an image of the subject's eye while the fixation target is projected onto the subject's eye involves illuminating the subject's eye using an infrared illumination source. In some embodiments, the infrared illumination source is different from the illumination source that is used in conjunction with the DMD to project the fixation target onto the subject's eye.

At act 1206, a processor is used to transmit instructions to the DMD configured to cause the DMD to vary the fixation target. Additionally or alternatively, the processor may be configured to analyze the image data from the imaging device. For example, the processor may be configured to determine the gaze angle, eye velocity, speed at which the eye responds to a stimulus (e.g., fixation target movement), among other metrics. This type of information can be useful in understanding how the eye is aging and detecting Alzheimer's. For example, response time may be slower in subjects with Alzheimer's.

### Computer Implementation

An illustrative implementation of a computer system 800 that may be used in connection with any of the embodiments of the technology described herein (e.g., such as one or more acts of the processes of FIG. 2, FIG. 9, FIG. 10, and/or FIG. 12) is shown in FIG. 8. The computer system 800 includes one or more processors 810 and one or more articles of manufacture that comprise non-transitory computer-readable storage media (e.g., memory 820 and one or more non-volatile storage media 830). The processor 810 may control writing data to and reading data from the memory 820 and the non-volatile storage device 830 in any suitable manner, as the aspects of the technology described herein are not limited to any particular techniques for writing or reading data. To perform any of the functionality described herein, the processor 810 may execute one or more processor-executable instructions stored in one or more non-transitory computer-readable storage media (e.g., the memory 820), which may serve as non-transitory computer-readable storage media storing processor-executable instructions for execution by the processor 810.

Computing device 800 may include a network input/output (I/O) interface 840 via which the computing device may communicate with other computing devices. Such computing devices may be interconnected by one or more networks in any suitable form, including a local area network or a wide area network, such as an enterprise network, an intelligent network (IN) or the Internet. Such networks may be based on any suitable technology and may operate according to any suitable protocol and may include wireless networks, wired networks or fiber optic networks.

Computing device 800 may also include one or more user I/O interfaces 850, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

Further, it should be appreciated that a computer may be embodied in any of a number of forms, such as a rack-mounted computer, a System on Module (SOM) computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures.

### CONCLUSION

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure. The acts performed as part of the methods may be ordered in any suitable way.

Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended. i.e., to mean including but not limited to. Only the transitional phrases “consisting of” and “consisting essentially of” shall be closed or semi-closed transitional phrases, respectively.

What is claimed is:

1. A system comprising:
a plurality of imaging components comprising:
an illumination source configured to emit light of a plurality of colors including a first color;
a digital micromirror device (DMD) configured to project illumination patterns onto a subject's eye using the light emitted from the illumination source;
at least one image sensor; and
a processor configured to perform a method for multispectral imaging, the method comprising:
transmitting instructions to one or more of the plurality of imaging components configured to cause the one or more of the plurality of imaging components to adjust an exposure to light of the first color relative to exposure to light of other colors included in the plurality of colors.

2. The system of claim 1, wherein the illumination source comprises one or more light emitting diodes (LEDs) and/or one or more lasers.

3. The system of claim 1, wherein transmitting the instructions to the one or more of the plurality of components comprises transmitting instructions to the illumination source configured to cause the illumination source to adjust an illumination intensity of the light of the first color.

4. The system of claim 1, wherein transmitting the instructions to the one or more of the plurality of components comprises transmitting instructions to the at least one image sensor configured to cause the at least one image sensor to adjust a gain associated with one or more pixels of the at least one image sensor.

5. The system of claim 1, wherein transmitting the instructions to the one or more of the plurality of components comprises transmitting first instructions to the DMD configured to cause the DMD to adjust a rate at which to project the illumination patterns onto the subject's eye and second instructions to the at least one image sensor configured to cause the at least one image sensor to adjust a shutter speed.

6. The system of claim 5, wherein the shutter speed corresponds to the rate at which the illumination patterns are projected onto the subject's eye.

7. A system comprising:
an illumination source configured to emit light;
a digital micromirror device (DMD) configured to project, during an imaging period, a plurality of illumination patterns onto a subject's eye using the light emitted from the illumination source, the imaging period starting at an initial time point corresponding to a projection of an initial illumination pattern onto the subject's eye and ending at a final time prior to a time of constriction of a pupil of the subject's eye;
a fundus imaging device configured to capture a plurality of images of a fundus of the subject's eye, each image in the plurality of images captured during projection of a respective illumination pattern of the plurality of illumination patterns; and
a processor configured to generate an image of the fundus using the plurality of images.

8. The system of claim 7, wherein the plurality of illumination patterns is a video sequence projected continuously onto the subject's eye during the imaging period.

9. The system of claim 8, wherein an intensity of the plurality of illumination patterns is less than or equal to a threshold intensity.

10. The system of claim 9, wherein the imaging period has a duration between 10 seconds and 40 seconds.

11. The system of claim 9, further comprising generating the image at least in part by combining at least some of the plurality of images using a super-resolution technique.

12. The system of claim 9, wherein the generated image has a higher resolution than respective resolutions of the at least some of the plurality of images.

13. The system of claim 9, wherein the imaging period has a duration between 5 and 150 milliseconds.

14. A method for imaging a fundus of a subject's eye using a fundus imaging system, the fundus imaging system comprising a fundus image sensor comprising a plurality of pixels and employing a rolling shutter and a digital micromirror device (DMD), the method comprising:
capturing an image of the fundus of the subject's eye at least in part by:
projecting a first illumination pattern onto a first portion of a subject's eye using the DMD; and
while projecting the first illumination pattern onto the first portion of the subject's eye, capturing image data depicting the first portion of the subject's eye using the rolling shutter and a first subset of the pixels of the fundus image sensor.

15. The method of claim 14, wherein capturing the image data depicting the first portion of the subject's eye using the rolling shutter comprising synchronizing the rolling shutter with the projection of the first illumination pattern onto the first portion of the subject's eye.

16. The method of claim 14, wherein capturing the image data depicting the first portion of the subject's eye using the first subset of pixels comprises exposing the first subset of pixels of the fundus image sensor, wherein the first subset of pixels are positioned along a same row or column.

17. The method of claim 16, wherein the first portion of the subject's eye is positioned along a rolling shutter integration line.

18. A system comprising:
at least one illumination source configured to emit light;
a digital micromirror device (DMD) configured to project a fixation target onto a subject's fundus using at least some of the light emitted from the illumination source;
an imaging device configured to capture an image of the subject's eye while the fixation target is projected onto the subject's fundus; and
a processor configured to transmit instructions to the DMD configured to cause the DMD to vary the fixation target.

19. The system of claim 18, wherein the at least one illumination source is further configured to emit infrared light that is projected onto the subject's eye while the fixation target is projected onto the subject's fundus.

20. The system of claim 19, wherein the imaging device is configured to capture the image of the subject's eye at least some of the infrared light that reflects from the subject's eye.
